(19) United States
(12) Patent Application Publication
(10) Pub. No.: US 2012/0232405 A1
(43) Pub. Date: Sep. 13, 2012

(54) COMPACT PROBE FOR TRACER-ASSISTED DIAGNOSTIC AND SURGERY

(76) Inventor: Eugene Grigoriev, Geneva, (CH)

(21) Appl. No.: 13/393,364

(22) PCT Filed: Aug. 29, 2009
(86) PCT No.: PCT/IB2009/053779

§ 371 Date: May 8, 2012

(51) Int. Cl. A61B 6/00

(52) U.S. Cl. 600/476; 600/407

(57) ABSTRACT

A simple and potentially disposable compact probe of FIG. 1, which is primarily aimed to be used for radio-guided or fluorescence-guided surgery, diagnostics or biopsy, and method of using it, is invented. The novel method reduces limitations inherent to the existing technologies. It consists in shifting most of the functions (signal analysis, detector controls and user visual and audio interfaces) from the probe to an external personal computer connected with the probe via wireless link. Maximum simplification and miniaturization of the probe itself makes it potentially disposable, supplied in sterile package similar to disposable syringe. Due to use of single photon time-resolved counting photo-sensors and microelectronic circuits with sub-nanosecond timing, the probe can be used with fluorescent markers as well. New probe should enhance efficiency of medical procedures and open opportunities for more wide application of the intra-operative probing techniques in medical practice, especially in oncology. Applications other than medical are also possible.

### STATE OF THE ART AND INVENTION DESCRIPTION

### State of the Art

Surgical radio-guided procedures have been used since a long time and consist in injecting patients with a radioactive isotope that emits b radiation (positrons and/or electrons) and/or g radiation and has the property of binding preferentially to the diseased tissues, e.g. the tumors, via their carrier molecules. The surgeon then uses a so-called ‘peroperative’ hand-held probe sensitive to the radioactivity emitted by the radio-isotope carrier molecules.

This type of radio-guided surgery has proven its efficiency and is commonly recognized and employed for the treatment of lung cancers, melanomas, thyroid cancer, neuroendocrine cancer and benign tumors such as, inter alia, parathyroid hyperplasias or osteoid osteomas. On the other hand, this radio-guided surgical technique using radiosensitive peroperative manual probes is still undergoing evaluation for applications in the treatment of tooth neck or colon cancers.

Radiosensitive peroperative manual probes are also of great value in the context of the operations known as ‘sentinel lymph node (SLN) biopsy’. This cancer diagnostic technique is based on the sentinel node concept, according to which the state of the sentinel lymph node of the nodal regional lymphatic basin draining a primary tumor is an indication of the cancerous or non-cancerous state of the whole of the nodal lymphatic region in question. If the sentinel node is affected, the whole region is affected, and vice-versa.

Hitherto, the radiosensitive peroperative probes most widely used routinely for radio-guided surgery, especially SLN biopsy, have been g probes suitable for detecting g radiation (g ray or photon). However, g radiation has the disadvantage of a relatively long range within biological tissues, which creates a considerable background. It is thus difficult to differentiate the tumoral areas from the healthy tissues. Moreover, this contamination by the g radiation background makes it difficult, if not impossible, to detect small radio-labelled tumoral objects.

Peroperative manual probes sensitive to b radiation (positron and/or electron) have therefore been developed as possible alternatives to g probes.

Insofar as b particles have a relatively short range in tissues, peroperative manual probes whose principle is based on detecting these b emissions are potentially much more sensitive in the delimitation and location of focused cancerous areas than more standard probes which operate by the detection of highly penetrating g radiation.

Positron-emitting isotope markers with a high affinity for cancerous tissues are known, an example being ¹⁸F-labeled 2-fluorodeoxy-D-glucose (FDG). ¹⁸F-labeled FDG is a specific marker for a carbohydrate hyper metabolism indicative of malignant tissues or inflammatory tissues. This marker is already used in diagnostic medicine for mapping the spread of a cancer with the aid of complex and expensive positron-detecting equipment (PET (Positron Emission Tomography) camera). After diagnostic examination, the ¹⁸F-labeled FDG, which is still present in the tumors, can be used for guiding of surgery or biopsy tools towards the tumor. However, this technique has a serious disadvantage—it can be applied only shortly after PET-examination (due to fast decay of ¹⁸F) and can be used only in clinics with special equipment (PET scanners and all related radio-protection facilities). Therefore, replacing FDG or other radioactive markers by fluorescent ones can open much wider applications for this technique, reduce costs and eliminate all risks related to radioactivity.

Existing devices have several disadvantages, which create obstacles in more wide application of this technique: they are rather bulky, expensive, usually controlled from a remote control box via cable. Therefore, special measures have to be taken in operation theatre in order to preserve sterility (cleaning and sterilization before/after the operation, utilization of sterile protection sleeves, etc.) which reduce sensitivity (stop charged particles) and limit freedom of manipulation for the surgeon. Existing probes are sensitive only to radioactive tracers; there are no compact probes which are capable to detect single photons from fluorescent markers or auto-fluorescence.

### DESCRIPTION OF THE INVENTION

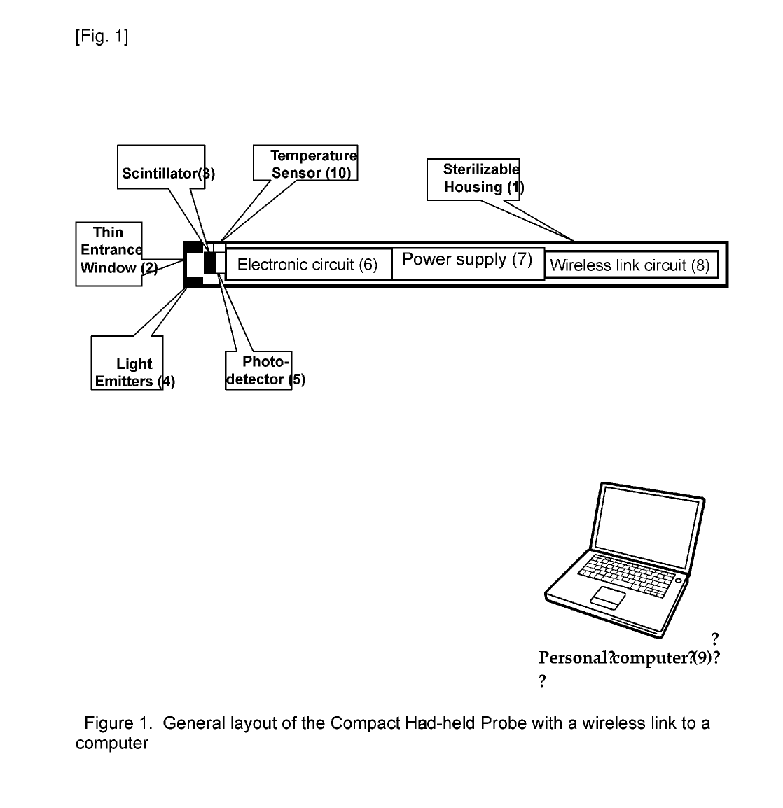

A new method (claim 1) and device (claims 2 and 3) is proposed which enhances efficiency of procedures using radioactive tracers and allows replace radioactive tracers by more cheap and safe fluorescent ones. The compact probe according to the invention is shown in FIG. 1. Preferred embodiments of the invention are listed in the dependent claims. The probe according to the invention is designed in a way which provides maximum convenience and simplicity in operation for the user, minimal weight and cost (potential disposability), preserving at the same time maximum performance. This is reached by introduction of several novelties.

The method consists in detection of signals emitted by tracers with time-resolved photon counting technique in a compact, simple and handy autonomous detector, which is supplied in a sterile packaging ready for operation, and transmitting those signals using wireless link to a remote computer (9), which performs most of the functions, such as: control, calibration, analysis, user interface via visual display and audio signals, etc.. Thus, the probe used in this method is a simple, cheap and potentially disposable device, carrying mainly detection functions, while all controls, analysis and display are performed externally. Moving most of the processing functions from the probe to the external control device (computer), and introduction of single-photon counting technique with sub-nanosecond timing, allow to enhance sensitivity, reduce size, weight and cost of the probe itself, thus leading to compactness, easy sterilization and disposability. Another novelty consists in introduction in the probe of a temperature-compensating circuit controlled from the external control device via wireless link. This ensures stability of operating parameters in applications such as surgery, where rapid and large temperature variations are possible. For application with fluorescent tracers the probe is equipped with a light-injector instead of scintillator. Altogether these features open opportunities to more wide application of the intra-operative probe techniques, which is widely recognized as very promising, in medical practice. Use of fluorescent markers instead of radioactive ones would allow medical establishments which are not utilizing methods of nuclear medicine, to profit as well from intra-operative probing techniques.

The device (compact probe) is assembled in a sterilizable housing (1) with a battery (8) and a thin front entrance window (2). The two possible embodiments for radioactive-tracer (a) and for fluorescent-tracer applications (b) are described below. They can be optionally combined in a single embodiment (multimodal probe).
1. a) Charged particles emitted by radioactive tracer or resulting from interaction of those with surrounding media, are detected by the scintillator (3) which converts ionization produced by those particle into photons of light. These photons are detected by photo-detector (5), which gives fast electrical signals on the output. These signals are amplified and digitized by the electronic circuit operating with sub-nanosecond precision (6) and sent by the transmitter/receiver (8) via wireless link to a remote computer (9), which analyses them using special software and gives visual and audio information to the user, thus providing guidance in operation. On the basis of this analysis the computer also sends controlling signals to the probe for calibration, adjustment and compensation for temperature variations.

1. b) Instead of (or along with) the scintillator (3) the device is equipped with pulsed light emitter(s) (2), which stimulate fluorescence of the fluorescence tracer or auto fluorescence of specific molecules of interest. Photons produced by fluorescence are detected by photo-detector (5), which gives fast electrical signals on the output. These signals are amplified and digitized by the electronic circuit operating with sub-nanosecond precision (6) and sent by the transmitter/receiver (8) via wireless link to a remote computer (9), which analyses them, using special software and gives visual and audio information to the user, thus providing guidance in operation. On the basis of this analysis the computer also sends controlling signals to the probe for calibration, adjustment and compensation for temperature variations.

Fast real-time temperature monitoring is performed either with a miniature temperature sensor (10) or by analysing the signals received from the photo-detector. In the latter case the photo-detector (5) serves also as a temperature sensor. Compensation for temperature variations is done by changing operating voltages generated by DC-converters of the electronic circuit (6) or by changing operating parameters of the electronic circuit itself.

1. A method of identification and localization of clusters with abnormal concentrations of radioactive or fluorescent substances using compact hand-held autonomous probe, which performs merely detection functions and is connected by wireless link to an external device, which performs most of control, calibration, analysis and human interface functions.

2. A compact hand-held probe comprising: a photo-detector capable to count single photons of light; an autonomous power supply; and an electronic circuit with the following functions: generating necessary DC voltages; receiving signals from the photo-detector and analyzing pulse-height and time characteristics of those; converting and transmitting the signals to an external device using wireless connection;
receiving control signals from the external control device via wireless connection.

3. A compact hand-held probe comprising a photo-detector capable to count single photons of light; an electronic circuit with light-emitter which illuminates the zone viewed by the photo-detector with fast light pulses; an autonomous power supply; and an electronic circuit with the following functions: generating necessary DC voltages; receiving signals from the photo-detector and analyzing pulse-height and time characteristics of those; converting and transmitting the signals to an external device using wireless connection; receiving control signals from the external control device via wireless connection.

4. The compact hand-held probe according to claim 2, wherein the photo-detector is optically coupled to one or several pieces of organic or inorganic scintillating material, which converts energy of particles emitted by radioactive substances into light.

5. The compact hand-held probe according to claim 3, wherein the photo-detector is optically coupled to one or several pieces of organic or inorganic scintillating material, which converts energy of particles emitted by radioactive substances into light.

6. The compact hand-held probe according to claim 2, wherein the photo-detector is optically coupled to a mask of optical filters and/or optical lenses which concentrate light on photosensitive zones and/or select particular wavelengths.

7. The compact hand-held probe according to claim 3, wherein the photo-detector is optically coupled to a mask of optical filters and/or optical lenses which concentrate light on photosensitive zones and/or select particular wavelengths.

8. The compact hand-held probe according to claim 2, wherein the photo-detector is composed by several photon-counting elements.

9. The compact hand-held probe according to claim 3, wherein the photo-detector is composed by several photon-counting elements.

10. The compact hand-held probe according to claim 2, wherein the photo-detector is single-channel or multichannel silicon circuit containing avalanche photodiodes operating above breakdown voltage (‘Geiger mode operation’).

11. The compact hand-held probe according to claim 3, wherein the photo-detector is single-channel or multichannel silicon circuit containing avalanche photodiodes operating above breakdown voltage (‘Geiger mode operation’).

12. The compact hand-held probe according to claim 2, which is aimed for single-use (disposable) and supplied in sterile package satisfying standards acceptable in operation room.

13. The compact hand-held probe according to claim 3, which is aimed for single-use (disposable) and supplied in sterile package satisfying standards acceptable in operation room.

14. The compact hand-held probe according to claim 2, which contains a temperature sensor in the vicinity of the photo-detector for dynamic correction of operating parameters according to temperature variations.

15. The compact hand-held probe according to claim 3, which contains a temperature sensor in the vicinity of the photo-detector for dynamic correction of operating parameters according to temperature variations.
